(19) United States
(12) Patent Application Publication
(10) Pub. No.: US 2025/0272223 A1
(43) Pub. Date: Aug. 28, 2025

(54) SYSTEM AND METHOD FOR SMART DEVICE MEMORY ASSISTANCE

(71) Applicant: Hilton Nicholson, Bossier City, LA (US)

(72) Inventor: Hilton Nicholson, Bossier City, LA (US)

(21) Appl. No.: 19/049,650

(22) Filed: Feb. 10, 2025

Related U.S. Application Data
(60) Provisional application No. 63/558,987, filed on Feb. 28, 2024.

(51) Int. Cl. G06F 12/02

(52) U.S. Cl. CPC G06F 12/0223

(57) ABSTRACT

A system and method for smart device memory assistance are provided. The system of the present invention includes a processing device, an input/output device, and at least one peripheral device. The Input/Output device of the present invention monitors an environment of a user and provides data items from the environment of the user to the processing device. The processing device analyzes the data items and provides outputs to the Input/Output device to aid the user in life tasks.

### CROSS-REFERENCE TO RELATED APPLICATIONS

This application claims the benefit of priority of U.S. provisional application No. 63/558,987, filed Feb. 28, 2024, the contents of which are herein incorporated by reference.

### BACKGROUND OF THE INVENTION

### Field of Endeavor

The present invention relates to smart assistants, and more particularly, to a system and method for providing memory assistance from a smart device.

### Background and Related Art

People with cognitive memory impairment, or decline, such as Alzheimer's disease and various forms of Dementia, have difficulties remembering people and basic life activities such as eating, taking medicines, utilizing appliances, finding their way home, etc. The issues range from frustrating for the person suffering, family, and friends, to potentially life threatening if medications, or meals, are routinely missed.

Currently, caregivers are required to assist, or otherwise remind a person with cognitive impairment in remembering peoples' names/faces, and other basic life activities. However, caregivers are not always able to assist, and determine if basic life activities have gone unfulfilled, which can lead to accelerated loss of cognitive function and personal autonomy.

As can be seen, there is a need for a system and method for providing memory assistance without loss of personal autonomy.

### BRIEF DESCRIPTION OF THE DRAWINGS

FIG. 1 is a diagram of an embodiment of a Smart Device System, in accordance with aspects of the present invention; and

FIG. 2 is a flowchart of an embodiment of a method of operation of a Smart Device System, according to aspects of the present invention.

### SUMMARY OF THE INVENTION

In the present invention a system and method for memory assistance are provided. The memory assistance system of the present invention comprises input/output devices and a processing device, having one or more modules configured to assist a user. In an aspect of the present invention the processing device includes at least one memory storing instructions; and at least one processor configured to execute the instructions to perform a method. The method of the processing device receives one or more data items from the input/output devices via one or more sensors of the input/output device. The one or more data items are analyzed by one or more modules of the processing device to extract one or more information items, such as, but not limited to, facial recognition information, speech recognition information, motion recognition information, and/or peripheral device information, such glucose level information. The one or more information items is compared to one or more records in a data storage to determine if a match exists, and if so, at least one metadata item, such as but not limited to, a name, a relationship indication, one or more instructions, or other textual information, is returned to the processing device. The at least one metadata item is transmitted to the input/output device for output to the user. If no match exists between the one or more information items, and the one or more records a new record can be created using the at least one information item, and additional information can be input and stored in the new record in association with the one or more information items.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

More than 55 million people worldwide have cognitive memory impairments such as Alzheimer's disease, or dementia. People with cognitive memory impairments struggle with identification of relatives, or acquaintances, and with providing for their own basic daily needs, such as nutrition and medication compliance. These struggles lead quickly to loss of autonomy and independence, which can significantly shorten their lifespan.

Broadly, an embodiment of a system the present invention may include one or more servers and at least one computer. Each server and computer of the present invention may each include computing systems. This disclosure contemplates any suitable number of computing systems. This disclosure contemplates the computing system taking any suitable physical form. As example and not by way of limitation, the computing system may be a virtual machine (VM), an embedded computing system, a system-on-chip (SOC), a single-board computing system (SBC) (e.g., a computer-on-module (COM) or system-on-module (SOM)), a desktop computing system, a laptop or notebook computing system, a smart phone, smart glasses, smart watch, smart device, an interactive kiosk, a mainframe, a mesh of computing systems, a server, an application server, or a combination of two or more of these. Where appropriate, the computing systems may include one or more computing systems; be unitary or distributed; span multiple locations; span multiple machines; or reside in a cloud, which may include one or more cloud components in one or more networks. Where appropriate, one or more computing systems may perform without substantial spatial or temporal limitation one or more steps of one or more methods described or illustrated herein. As an example, and not by way of limitation, one or more computing systems may perform in real time or in batch mode one or more steps of one or more methods described or illustrated herein. One or more computing systems may perform at different times or at different locations one or more steps of one or more methods described or illustrated herein, where appropriate.

In certain embodiments, the network may refer to any interconnecting system capable of transmitting audio, video, signals, data, messages, or any combination of the preceding. The network may include all or a portion of a public switched telephone network (PSTN), a public or private data network, a local area network (LAN), a metropolitan area network (MAN), a wide area network (WAN), a local, regional, or global communication or computer network such as the Internet, a wireline or wireless network, an enterprise intranet, or any other suitable communication link, including combinations thereof.

In some embodiments, the computing systems may execute any suitable operating system such as IBM's zSeries/Operating System (z/OS), MS-DOS, PC-DOS, MAC-OS, WINDOWS, UNIX, OpenVMS, an operating system based on LINUX, or any other appropriate operating system, including future operating systems. In some embodiments, the computing systems may be a web server running web server applications such as Apache, Microsoft's Internet Information Server™, and the like.

In particular embodiments, the computing systems includes a processor, a memory, a user interface and a communication interface. In particular embodiments, the processor includes hardware for executing instructions, such as those making up a computer program. The memory includes main memory for storing instructions such as computer program(s) for the processor to execute, or data for processor to operate on. The memory may include mass storage for data and instructions such as the computer program. As an example, and not by way of limitation, the memory may include an HDD, a floppy disk drive, flash memory, an optical disc, a magneto-optical disc, magnetic tape, a Universal Serial Bus (USB) drive, a solid-state drive (SSD), or a combination of two or more of these. The memory may include removable or non-removable (or fixed) media, where appropriate. The memory may be internal or external to computing system, where appropriate. In particular embodiments, the memory is non-volatile, solid-state memory.

The user interface includes hardware, software, or both providing one or more interfaces for communication between a person and the computer systems. As an example, and not by way of limitation, a user interface device may include a keyboard, keypad, microphone, monitor, mouse, printer, scanner, speaker, still camera, stylus, tablet, touchscreen, trackball, video camera, another suitable user interface or a combination of two or more of these. A user interface may include one or more sensors. This disclosure contemplates any suitable user interface and any suitable user interfaces for them.

The communication interface includes hardware, software, or both providing one or more interfaces for communication (e.g., packet-based communication) between the computing systems over the network. As an example, and not by way of limitation, the communication interface may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface. As an example and not by way of limitation, the computing systems may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, the computing systems may communicate with a wireless PAN (WPAN) (e.g., a BLUETOOTH WPAN), a WI-FI network, a WI-MAX network, a cellular telephone network (e.g., a Global System for Mobile Communications (GSM) network), or other suitable wireless network or a combination of two or more of these. The computing systems may include any suitable communication interface for any of these networks, where appropriate.

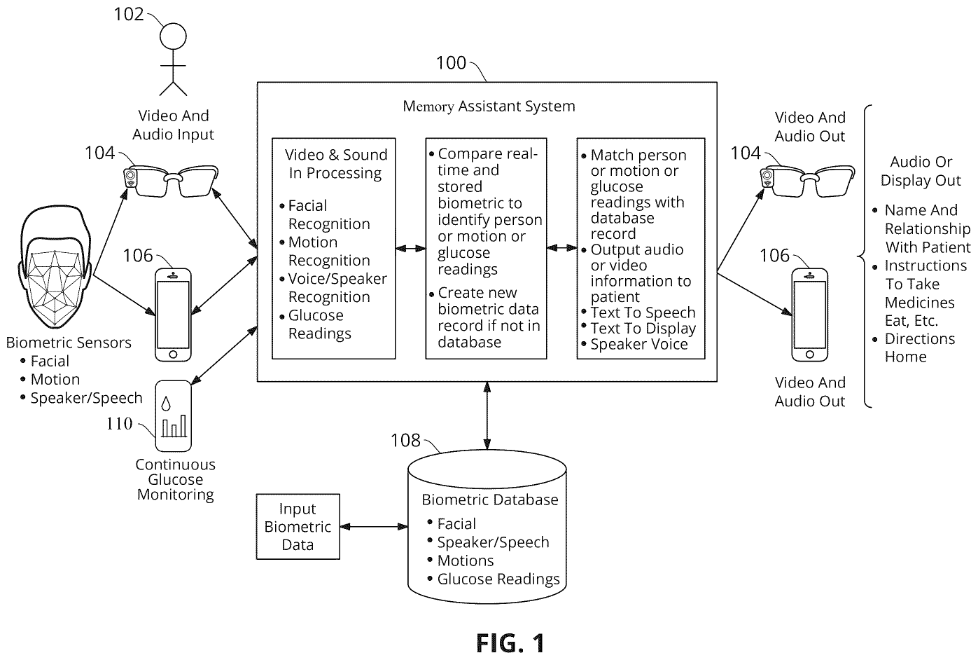

Referring now to FIG. 1, Memory Assistant System 100 is illustrated, according to aspects of the present invention. In embodiments, Memory Assistant System 100 can be one or more software applications, modules, programs, process, etc. resident on one or more computing systems, such as Input/Output device 104, processing device 106 and peripheral device 110, such as a glucose monitor. A user 102 can wear, or otherwise have in their possession, an input/output device 104, which can be adapted to receiving input from the environment and providing output to user 102. Input/Output device 104 can be communicatively coupled to processing device 106 and/or peripheral device 110, such as a glucose monitor, via one or more communication interfaces. Processing device 106 and peripheral device 110 can be adapted for processing of environmental input from Input/Output device 104 and providing output to Input/Output device 104. Memory Assistant System 100 can include data storage 108, which can be adapted to store parameters used by Memory Assistant System 100 for input/output processing, and security. In an embodiment, data storage 108 can be a memory of Input/Output Device 104, Processing Device 106 and peripheral device 110, or can be a dedicated computing system with memory and can include biometric data, Identification data, medical information, notifications, reminders, instructions, kinesiological data, glucose information, and/or any data useful for assisting user 102 with interacting with their environment.

In an exemplary embodiment, Input/Output device 104, as a computing system, can have components associated therewith, described above, such as memory, processor, integrated camera, one or more sensors, an audio interface, and one or more user interfaces. In one embodiment, Input/Output device 104 can be a smart device such as smart glasses, a smart watch, and/or other wearable devices.

In an exemplary embodiment, Processing Device 106, as a computing system, can have components associated therewith, described above, such as a memory, a processor, an integrated camera, an audio interface, one or more additional interfaces, and one or more user interfaces. In one embodiment, Processing device 106 can be a smart phone with memory, processor, integrated camera, one or more sensors, an audio interface, and one or more user interfaces. In one, embodiment, processing device 106 can be a smart phone, laptop computer, computer, smart watch, and/or other mobile processing device. It is understood that Input/Output device 104 and Processing device 106 have been described as separate devices, with Memory Assistant System 100 resident on processing device 106, however, it is equally contemplated that Input/Output Device 104 and Processing Device 106 can be the same device housing the functionality of Memory Assistant System 100 and performing the functionality associated therewith.

In an exemplary embodiment, peripheral Device 110, as a computing system, can have components associated therewith, described above, such as a memory, a processor, an integrated glucose monitor. In one embodiment, peripheral device 110 can be a smart glucose monitor with memory, processor, one or more sensors, and one or more user interfaces. In one, embodiment, peripheral device 110 can be an intrusive or non-intrusive glucose monitor or smart watch, and/or other mobile processing device.

In embodiments, data storage 108 can any data store known in the art which can store one or more records associated with user 102. In embodiments, each record of the one or more records can include a unique identifier, and one or more metadata items associated with the unique identifier. In embodiments, the unique identifier for each record can be one of: a facial recognition identifier, a speaker recognition identifier, a motion identifier, and/or a user identifier. Additionally, the one or more metadata items associated with the unique identifier can be one or more of: a name of a person, a relationship indicator, and/or one or more additional information items. In embodiments, the one or more additional items can be one or more of: one or more instructions or directions, one or more thresholds, and/or one or more plain text items. In embodiments, the one or more one or more records can be added to data storage 108 by a user, such as user 102, or another user, such as a caregiver, or can automatically added by Memory Assistant System 100 upon initial recognition.

Referring now to one or more functionalities of Memory Assistant System 100. In embodiments, Memory Assistant System 100 can include one or more software applications, modules, programs, process, etc., configured to perform one or more functions, such as, but not limited to: facial recognition, motion recognition, speech recognition, and/or peripheral device interfacing module, such as retrieving one or more data items from peripheral device, i.e. glucose readings from a glucose monitor.

In embodiments, a facial recognition module can be configured to recognize one or more faces of person(s). In embodiments, the facial recognition module can receive one or more image items, such as real-time streams, or batch files, containing one or more faces, or facial information from Input/Output device 104. In embodiments, the one or more image items can include any known format for multimedia, or streaming files. In embodiments, one or more sensors, such as a still camera, video camera, or other image capturing device of Input/Output device 104 can transmit the one or more image items to Processing Device 106, running the facial recognition module.

In embodiments, the facial recognition module can analyze the one or more image items to determine one or more facial identifiers, such as a faceprint, fingerprint, etc. The facial recognition module can compare the one or more facial identifiers to one or more identifiers stored in data storage 108 for potential matches. In embodiments, a record corresponding to the matched one or more identifiers is returned to facial recognition module, in the event of a match, and is transmitted to Input/Output device 104. In embodiments, if no match is found facial recognition module can prompt a user to enter one or more metadata items associated with the one or more facial identifiers. Once entered the facial recognition module can store the one or more facial identifiers as an identifier of a new record in data storage 108 along with the one or more metadata items entered by the user.

In embodiments, a motion recognition module can be configured to recognize one or more movements of user 102. In embodiments, motion recognition module can monitor, using one or more sensors of Input/Output Device 104, one or more movements of user 102, in real-time or through batch processing, to determine compliance with one or more movement items. In embodiments, the one or more movements can be captured in any known format for multimedia, or streaming files, or using other sensor technologies. In embodiments, one or more sensors, such as a still camera, video camera, or other motion capturing device of Input/Output device 104 can transmit the one or more motions of user 102 to Processing Device 106, running the motion recognition module.

In embodiments, motion recognition module can analyze the one or more motions of user 102 to determine one or more motion identifiers. The motion recognition module can compare the one or more motion identifiers to one or more identifiers stored in data storage 108 for potential matches.

In embodiments, the peripheral device monitoring module, as a glucose monitoring module, can analyze the one or more glucose readings from the peripheral device 110, as a Continuous Glucose Monitoring device, to determine if meals are being eaten at schedule intervals. The glucose monitor module can compare the one or more glucose readings to one or more glucose readings stored in data storage 108 for potential matches. In embodiments, the one or more glucose readings can be indicative of thresholds, such that a reading below and/or above the threshold can indicate that a user has not eaten a meal. For example, the one or more glucose readings can show low glucose in user 102, such that user 102 has missed one or more meals. In embodiments, an audio prompt via device 106 is provide to user 102 to eat scheduled meals. After a predetermine interval after audio prompt, one or more identifiers is returned to glucose monitor module, in the event of no match (meal not eaten), and is transmitted to Input/Output device 104. In embodiments, if match is found (meal eaten) no action is taken.

In embodiments, a speech recognition module can be configured to recognize one or more person(s) by speech. In embodiments, the speech recognition module can receive one or more speech items, such as real-time streams, or batch files, containing one or more audio data, or speech information from Input/Output device 104. In embodiments, the one or more speech items can include any known format for multimedia, or streaming files. In embodiments, one or more sensors, such as a microphone, or other audio capturing device of Input/Output device 104 can transmit the one or more speech items to Processing Device 106, running the speech recognition module. In embodiments, a record corresponding to the matched one or more identifiers is returned to motion recognition module, in the event of a match, and is transmitted to Input/Output device 104. In embodiments, if no match is found motion recognition module can prompt user 102 to perform one or more actions or instructions.

In embodiments, the speech recognition module can analyze the one or more speech items to determine one or more audio identifiers, such as an audio print, fingerprint, etc. The speech recognition module can compare the one or more speech identifiers to one or more identifiers stored in data storage 108 for potential matches. In embodiments, a record corresponding to the matched one or more identifiers is returned to speech recognition module, in the event of a match, and is transmitted to Input/Output device 104. In embodiments, if no match is found speech recognition module can prompt a user to enter one or more metadata items associated with the one or more speech identifiers. Once entered the speech recognition module can store the one or more speech identifiers as an identifier of a new record in data storage 108 along with the one or more metadata items entered by the user.

In embodiments, a peripheral device interfacing module can be configured to interface with one or more peripheral devices. In embodiments, a peripheral device interfacing module can interface with one or more peripheral devices to receive one or more data items associated with user 102. In embodiments, the one or more data items can be received in real-time, or through batch processing, and can be used for comparison with one or more records in data storage 108. In embodiments, the one or more data items can be compared to one or more metadata items of the one or more records. For example, the one or more data items can be glucose readings of user 102, which can be compared to one or more glucose thresholds associated with user 102. In embodiments, a record corresponding to the matched one or more data items is returned to speech recognition module, in the event of a match, and is transmitted to Input/Output device 104.

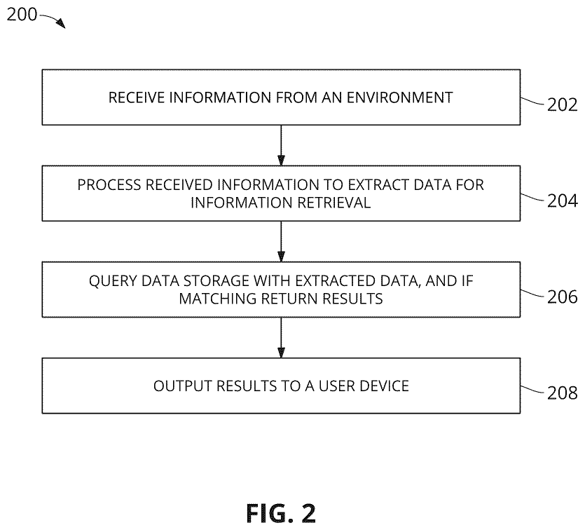

Referring now to FIG. 2, method 200 of operation of Memory Assistant System 100 is illustrated. In embodiments, method 200 can operate using one or more of the modules described above with respect to Memory Assistant System 100 operating on processing device 106. In embodiments, method 200 operates as user 102 wears Input/Output Device 104, which is communicatively coupled to processing device 106 and peripheral device 110.

At step 202, one or more data items is received from Input/Output device 104. In an embodiment, content can be received through one or more interfaces of Input/Output device 104, such as a camera, a microphone, or other sensors, such as interfaces associated with one or more peripheral devices. In embodiments, content can include multi-media content related to an individual or individuals, such as image, video, or audio content, an environmental object, a peripheral device reading, and/or an action/inaction of user 102.

At step 204, the one or more data items are processed to extract one or more information items for information retrieval by processing device 106. In embodiments, the one or more data items can be transmitted to one or more modules, such as facial recognition module, motion recognition module, speech recognition module, and/or peripheral device interfacing module, resident on Memory Assistant System 100 in processing device 106 to process and extract the one or more information items necessary for each module. In embodiments, the one or more information items can be extracted through one or more modules, such as facial recognition module, motion recognition module, speech recognition module, and/or peripheral device interfacing module, resident on Memory Assistant System 100, as the analysis process of the one or more modules. For example, the one or more data items can be provided to: the facial recognition module as the one or more image items analyzed to determine one or more facial identifiers; the motion recognition module as the one or more motions of user 102 to determine one or more motion identifiers; the speech recognition module as the one or more speech items to determine one or more audio identifiers; and/or peripheral device interface for extraction.

At step 206, the one or more information items can be used to query data storage 108, or other storage device(s), from processing device 106 for one or more results. In embodiments, the query can be a comparison of the one or more information items with one or more unique identifiers and/or one or more metadata of the one or more records of data storage 108. In the event that the one or more information items matches the one or more unique identifiers and/or one or more metadata of the one or more records, one or more output items can be returned. In embodiments, the one or more output items can be the one or more metadata of the matched one or more records. In the event that there is no match between the one the one or more information items and the one or more unique identifiers and/or one or more metadata of the one or more records, a new record can be created using the one or more information items.

In embodiments, the new record can store the one or more information items as either the unique identifier or one or more metadata items, and additionally, prompts can be provided to add one or more additional user input items as one or more metadata items. For example, in the case that the one or more information items is one of: a facial identifier, or a speech identifier, the one or more information item can be stored as the unique identifier for the new record, and one or more prompts can be provided to enter additional information storable as the one or more metadata items, such as a name of the person, a relationship of the person to user 102, and/or one or more textual items. In another example, in the case that the one or more information items is one or more data items from a peripheral device, the one or more information items can be stored as one or more metadata associated with a unique identifier matching user 102.

At step 208, processing device 106 can provide the one or more output items to Input/Output device 104 for display. In embodiments, either processing device 106 or Input/Output Device 104 can include one or more modules, applications, etc., to convert the one or more output items to a different format. In embodiments, the one or more output items can be output to a graphical display of Input/Ouput device 104, or alternatively as one or more differing outputs, such as text-to-speech.

To further aid understanding, exemplary operations of Smart Device System 100 are illustrated below. At a point prior to use a caregiver, or other user, can upload one or more records to data storage 108 for use in Memory Assistant System 100. In a first exemplary operation, user 102 can be in a familial setting with an individual(s). Input/Output device 104 can process the environment to one or more data items, such as images, video, and sound, which can be sent to and processed by processing device 106. In this exemplary operation, content can include faces, and/or identifying features, of the individual(s). Processing device 106 can perform image processing and/or audio processing, for facial recognition, motion recognition, or speech recognition, to determine the one or more information items needed to query data storage 108. Using the one or more information items, processing device 106 can query data storage 108 to return one or more records for each matched one or more information item. The records can be sent to Input/Output device 104 for display such that user 102 could recognize and interact with the individual(s).

In a second exemplary operation, user 102 can be in a solitary environment, such as a housing unit. User 102 can be wearing input/output device 104 which can be actively monitoring an environment of user 102. Specifically, input/output device 104 can monitor motions/movements of user 102 to determine compliance with daily regimens, such as eating, and medication compliance. Input/output device 104 can periodically send captured environmental data to processing device 106, which can utilize image processing, like motion capture, to determine if user 102 is eating and/or complying with medication requirements. Processing device 106 can utilize environmental data to query data storage 108 for medication information and/or eating schedule to determine user's 102 compliance. In case of non-compliance, processing device 106 can send a notification, reminder, or other message, to input/output device 104 that can be displayed to user 102. Additionally, processing device 106 can send a notification, reminder, or other message, to external individuals such as a caregiver, warning of non-compliance. It is understood that the above operations are exemplary, and additional functionalities are contemplated without departing from the scope of the invention.

It should be understood, of course, that the foregoing relates to exemplary embodiments of the invention and that modifications may be made without departing from the spirit and scope of the invention as set forth in the following claims.

What is claimed is:

1. A system, comprising:
at least one memory storing instructions; and at least one processor configured to execute the instructions to perform a method, the method comprising:
receiving, from a first device, one or more data items;
analyzing the one or more data items to extract one or more information items;
determining if a match exists between the one or more information items and one or more records;
if a match exists, returning at least one metadata item associated with the one or more records;
outputting, to the first device, the at least one metadata item; and
if no match exists, creating, in the datastore, a record using the at least one information item.

2. The system of claim 1, wherein the at least one data item is one of: multi-media data, environmental data, image data, voice data, biometric data, glucose data, sound data, or video data.

3. The system of claim 1, wherein analyzing further comprises:
analyzing the one or more data items by one or more of:
facial recognition module configured to analyze the one or more data items to determine one or more facial identifiers;
a motion recognition module configured to recognize one or more movements of a user;
a speech recognition module configured to recognize one or more users by speech; or
a peripheral device interfacing module configured to interface with one or more peripheral devices.

4. The system of claim 3, wherein the peripheral device interfacing module is a glucose module configured to recognize if meals are being consumed within predefined intervals.
